(19) United States
(12) Patent Application Publication
(10) Pub. No.: US 2011/0284653 A1
(43) Pub. Date: Nov. 24, 2011

(54) Adaptive Emanator of a Fluid

(75) Inventors: Martin Butler, Hull (GB); Chris Jones, Montvale, NJ (US); Kate Langley, Hull (GB); Shaun Rymer, Hull (GB)

(73) Assignee: Reckitt & Colman (Overseas) Limited, Slough, Berkshire (GB)

(21) Appl. No.: 12/741,155

(22) PCT Filed: Nov. 7, 2008
(86) PCT No.: PCT/GB08/03762

§ 371 Date: May 25, 2011

(30) Foreign Application Priority Data
Nov. 7, 2007 (GB) 0721829.0

(51) Int. Cl. A61L 9/04

(52) U.S. Cl. 239/34

(57) ABSTRACT

The present invention describes an emanation device that is adaptive to its surrounding environment. In one embodiment this device includes a housing adapted to receive a container of fluid and sensor means provided by at least one motion sensor means and/or at least one odour sensor means, emanation means adapted to emanate a fluid, a controller in communication with said sensor means and said emanation means, wherein the controller is operative to analyse the input from the sensor means and make a determination of the quantity of fluid to emanated in response to said input and instruct the emanation means to emanate the determined quantity of fluid.

### FIELD OF THE INVENTION

The present invention relates to an emanation device that is configured to adapt the emanation of a fluid into the surrounding environment based on a determination of the characteristics of the surrounding environment and particularly, but not exclusively, for the emanation of fluids such as fragrances, deodorizing and/or pest control materials.

### BACKGROUND

Devices are known in which a bottle of volatile liquid has a upwardly projecting wick and a heater is located in the vicinity of the upper end of the wick to accelerate the evaporation of volatile liquid from the wick. The bottle, wick and heater are retained within a housing which carries an electric plug. To operate the heater the device is plugged into a wall socket. Devices of this type commonly claim to allow control of the rate of evaporation of the volatile liquids, for example, by varying the distance between the heater and the wick.

Devices are also known in which an aerosol air freshener is held within an automatic spraying device. A powered mechanism actuates the valve on the aerosol to periodically emit a spray of the air freshener. Devices of this type commonly claim to allow control of the amount of spray over a fixed time period by the consumer being able to vary the time period between emissions. Such automatic spraying devices are typically unable to provide adjustment in response to external stimuli.

Known prior art devices suffer from the drawback of efficiency and convenience. Typically the user has to manually change the apparatus from “normal” to “boost” mode, and then to switch it back to “normal” mode when this effect is no longer necessary (e.g. when the room is empty, or at night). Given the typical location of sources of electrical supply on walls (at a low level near the floor) or placement of electrical devices, this makes the switching process inefficient and inconvenient.

There is a need, therefore, for a device which overcomes the defects of the prior art and provides a timely and efficient alteration of the quantity of volatile liquid supplied to a room in response to the external stimuli sensed by the device.

### SUMMARY OF INVENTION

According to a first aspect of the present invention there is provided therefore an emanation device comprising:

a housing adapted to receive a container of fluid;
sensor means provided by at least one motion sensor means and/or at least one odour sensor means;
emanation means adapted to emanate, in use, the fluid;
a controller in communication with said sensor means and said emanation means;
wherein the controller is operative to:
analyse the input from the sensor means;
make a determination of the quantity of fluid to emanated in response to said input; instruct the emanation means to emanate the determined quantity of fluid.

Preferably the sensor means comprises at least one motion sensor means and at least one odour sensor means.

Preferably the emanation means is provided in the form of at least one heater means and/or at least one electric fan means when the container to be used with the device comprises a wick having a proximal end region within the container and a distal end region above the container from which the fluid, in use of the device, is to be emanated.

Alternatively, the emanation means may be provided in the form of an actuation means when the container to be used with the device comprises fluid held under pressure within the container, said actuation means being operable to cause a valve in the container to be opened, thus permitting fluid to be sprayed.

The controller is preferably operative to provide an increased amount of emanation of fluid in response to the input from the sensor means informing the controller of an increased amount of movement and/or odour in the vicinity of the device. The input from the sensor means may be analysed by the controller relative to a base level. This base level may be:

calculated by the device after being operated in a particular environment for a period of time; constantly calculated such that the device is able modify the base level over time relative to the input it receives from the sensor means over time;
a pre-determined value that is stored by the controller; or
controlled by a user.

There may be more than one base level. One of said base levels may be selectable by a user depending on the environment in which they wish to use the device and/or the effect they wish to achieve.

Where the container to be used with the device holds the fluid under pressure, such as an aerosol, the controller is preferably operable to control the duration of time the actuation means holds the valve of the container open. For instance, where a standard actuation of an aerosol is to hold the valve open for 1 second, the controller may direct the actuation means to hold the valve open for a period of time greater than 1 second when the input received by the controller from the sensor means is indicative of large amounts of movement and/or odour in the vicinity of the device. Similarly, if the input received by the controller is indicative of a small amount of movement and/or odour in the vicinity of the device, the controller may direct the actuation means to hold the valve open for less than 1 second. The time period of 1 second is used for the purposes of illustration only.

Where the container to be used with the device provides a metered dose of fluid, such as a metered dose aerosol, the controller may be operational in a different manner to that described above. In this embodiment where a standard actuation is for the release of a single dose, the controller may be operable to direct the actuation means to hold the valve open to release a full dose or repeatedly open and close the valve to release several doses depending on the input received from the sensor means.

Where the container to be used with the device uses a wick to transport the fluid out of the container for emanation, the controller is preferably operable to control the duration of time the heater means and/or fan means are activated. For instance, where a standard activation time is in the order of 10 minutes, the controller may direct the activation to occur for a period of time greater than 10 minutes when the input received by the controller from the sensor means is indicative of large amounts of movement and/or odour in the vicinity of the device. Similarly, if the input received by the controller is indicative of a small amount of movement and/or odour in the vicinity of the device, the controller may direct the activation to occur for a period of time that is less than 10 minutes.

Alternatively or additionally, the controller may be operable to control the amount of heat applied by the heater means and/or whether the fan is operable depending on the input received from the sensor means. For instance, where the input received from the sensor means suggests a small amount of movement and/or odour in the vicinity of the device, the controller may be operable to instruct the heater means to impart a standard amount of heat toward the distal end of the wick (e.g. a 0-50% duty cycle). However, when the input is indicative of a moderate amount of movement and/or odour in the nearby vicinity, the controller may instruct the heater means to impart a standard amount of heat, or a greater than standard amount of heat (e.g. a 50-80% duty cycle) and/or also instruct the fan means to be operable. Then, when the received input is indicative of a large amount of movement and/or odour the controller may instruct the heater means to impart a greater amount of heat towards the wick than the standard amount (e.g. a 50-80% duty cycle) or a maximum amount of heat (e.g. 80-100% duty cycle) and/or the controller may also instruct the fan means to be operable.

The heater means may be operable to impart a varying amount of heat, in use, to the wick of a container of fluid by varying the power consumed; increased power consumption resulting in increased heat being imparted to the wick and vice versa. Alternatively or additionally, the variance of the heat may be imparted by varying the distance between the heater means an the wick. Alternatively or additionally, the variance of the heat may be imparted by varying the thickness of a heat insulation means between the heater means and the wick.

The motion sensor means and/or odour sensor means may take the form as described below in relation to the second aspect of the present invention.

According to a second aspect of the present invention there is provided therefore an emanation device comprising:

a housing adapted to receive a container of fluid;
sensor means provided by at least one motion sensor means and at least one odour sensor means;
emanation means adapted to emanate, in use, the fluid;
a controller in communication with said sensor means and said emanation means;
wherein the controller is operative to analyse the input from the sensor means and control the emanation means to emanate the determined amount of fluid.

Preferably the sensor means is provided by one motion sensor means and one odour sensor means.

The motion sensor means may be provided in the form of at least one of: an infrared (IR) sensor; a laser sensor; and a sound sensor. The IR sensor, which is preferably a passive IR sensor, may be operable to detect radiation in the infrared spectrum, thus be capable of detecting the presence of a person or an animal within the vicinity of the device. The laser sensor may be operable to emit one or more laser beams and be adapted to detect when an object breaks the one or more beams by moving across the beam(s), thus indicating the presence of a person or an animal within the vicinity of the device. The sound sensor may be operable to detect sound within the vicinity of the device and, preferably, once the detected sound exceeds a predefined level this is indicative of movement within the vicinity of the device.

The odour sensor means may be operable to detect common household odours (and the chemicals which constitute) these malodours. For example: kitchen malodour; bathroom malodour; tobacco smoke; pet odours; mould and/or mildew; body odour; fish; onions; garbage; fragrance from other products (such as detergents, polishes, cleaning products etc). To facilitate such detection the odour sensor means may be operable to detect at least some of the following chemical components: amines and nitrogen compounds; acids and/or sulphur compounds, such as mercaptans, thioacids, thioesters, sulfides, phenols and skatole.

Altering the quantity of fluid emanated by the device in response to the input received from the at least one motion means and/or at least one odour sensor means may be achieved as described above in relation to the first aspect of the present invention.

According to a third aspect of the present invention there is provided therefore an emanation device comprising:

a housing adapted to receive a container of fluid;
sensor means provided by at least one motion sensor means and/or at least one odour sensor means;
emanation means adapted to emanate, in use, the fluid;
a controller in communication with said sensor means and said emanation means;
wherein the controller is operative to control the emanation means to emanate fluid only when the controller has received an input from the sensor means that is indicative of movement and/or odour in the vicinity of the device.

A device according to the third aspect may be able to prevent the saturation of an environment, such as a room, with fluid. Advantageously this will permit the device to conserve fluid when not required and also conserve power consumption as the emanation means would not be activated until required.

Preferably the sensor means comprises at least one motion sensor means and at least one odour sensor means.

Preferably the emanation means is provided in the form of at least one heater means and/or at least one electric fan means when the container to be used with the device comprises a wick having a proximal end region within the container and a distal end region above the container from which the fluid, in use of the device, is to be emanated.

Alternatively, the emanation means may be provided in the form of an actuation means when the container to be used with the device comprises fluid held under pressure within the container, said actuation means being operable to cause a valve in the container to be opened, thus permitting fluid to be sprayed.

The controller is preferably operative to provide an increased amount of emanation of fluid in response to the input from the sensor means informing the controller of an increased amount of movement and/or odour in the vicinity of the device.

Altering the quantity of fluid emanated by the device in response to the input received from the at least one motion means and/or at least one odour sensor means may be achieved as described above in relation to the first aspect of the present invention.

The motion sensor means and/or odour sensor means may take the form as described above in relation to the second aspect of the present invention.

According to a fourth aspect of the present invention there is provided therefore an emanation device comprising:

a housing adapted to receive at least two separate fluid sources;
sensor means provided by at least one motion sensor means and/or at least one odour sensor means;
at least one emanation means adapted to emanate, in use, the fluid;
a controller in communication with said sensor means and said emanation means;
wherein the controller is operative to:
analyse the input from the sensor means;
make a determination of which source of fluid and/or the quantity of said fluid source to be emanated in response to said input;
instruct the emanation means to emanate the determined quantity of fluid.

Preferably the two or more fluid sources are different to each other. For instance, where there are two separate fluid sources, one source could be a fragrance and the other could be a deodorizing agent.

The controller may be operable to analyse the input from the sensor means to determine whether there is an increased amount of movement and/or odour in the vicinity of the device. The input from the sensor means may be analysed by the controller relative to a base level.

The controller may be operable to instruct the emanation means to alternate between the sources of fluids based on the amount of movement and/or odour detected. In one embodiment, where a high amount of movement and/or odour is detected, the controller may cause a first source of fluid to be emanated. Whereas where a low amount of movement and/or odour is detected, the controller may cause a second source of fluid to be emanated. For instance, in this embodiment the first source of fluid could be an a deodorizing agent which may be useful to attack the smells produced when large amounts of movement occur or lots of odour is present. Whereas, the second source of fluid may be a fragrance which may be useful to provide an agreeable perfume in the vicinity of the device without needing to attack the odours produced by a large amount of movement or the presence of a large amount of odour.

Where the device is provided with at least one motion sensor means and at least one odour sensor means, the controller may be operable to cause a first source of fluid to be emanated in response to an input received from the odour sensor(s) and may be operable to cause a second source of fluid to be emanated in response to an input received from the motion sensor(s). For instance, in this embodiment the first source of fluid may be an a deodorizing agent which may be useful to attack the odour detected by the odour sensor means and the second source of fluid may be a fragrance to provide an agreeable perfume in the vicinity of the device when motion is detected by the motion sensor means.

Altering the quantity of fluid emanated by the device in response to the input received from the at least one motion means and/or at least one odour sensor means may be achieved as described above in relation to the first aspect of the present invention.

The motion sensor means and/or odour sensor means may take the form as described above in relation to the second aspect of the present invention.

The device of any of the above-mentioned aspects may be provided with an indicator wherein said indicator is operable to indicate to a user what function the device is currently performing. The indicator may be operable to provide a visual indication and/or provide an audible indication.

Preferably the indicator is configured to provide a visual indication by emitting light from one or more light sources, preferably one or more LEDs.

The one or more light sources may be adapted to emit a different colour of light to indicate the current function the device is performing. Additionally or alternatively, the one or more light sources may blink or flash to indicate the current function the device is performing.

Alternatively or additionally, the device may be operable to visually indicate the function currently being performed by the device via a screen. The screen may be an LCD screen that is adapted to provide a message to a user, for instance such messages could include “ON”, “SENSING”, “MOTION DETECTED”, “RESTING”, “NORMAL MODE”, “DETECTION MODE”, “OFF”.

The device may be provided with a boost mechanism. The boost mechanism may be linked to a user operated switch or button or the like. On operating the boost mechanism the actuation means may actuate, and this actuation may occur regardless of the current mode of operation of the device. In effect, the boost mechanism may provide the user with a control to override the operation of the device for a single emanation.

The device may be power by mains-supplied electricity and/or be battery powered and/or be powered by solar cells located on the device. Most preferably the device is battery powered.

### BRIEF DESCRIPTION OF THE DRAWINGS

Embodiments of the invention will now be described, by way of example only, with reference to the following drawings in which:

FIG. 1 illustrates a perspective view of a device of the present invention; and

FIG. 2 illustrates a sectioned side view of a device of the present invention.

FIG. 3 illustrates a front elevation of an alternative device of the present invention with a front cover removed; and

FIG. 4. illustrates a front elevation of the alternative device with the front cover in place.

### DESCRIPTION OF AN EMBODIMENT

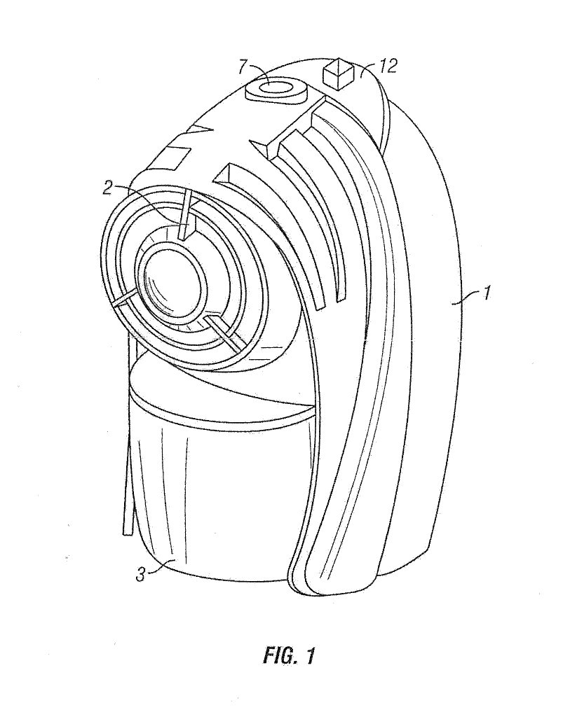

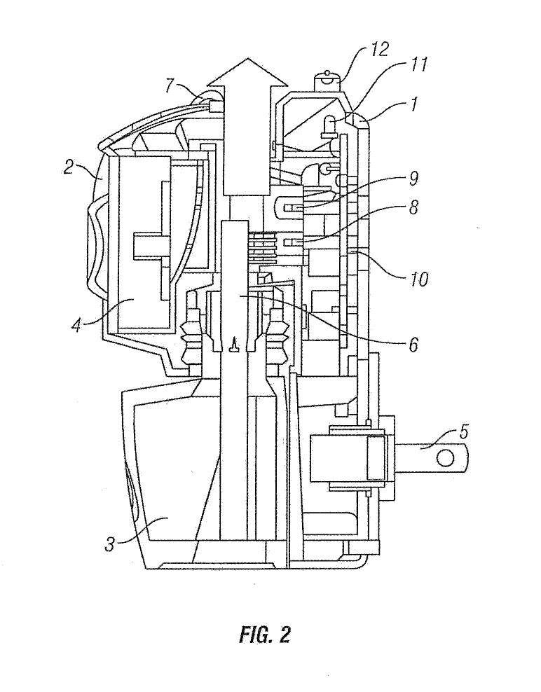

In general terms, the device 1 illustrated in FIGS. 1 & 2 is shown with a container of volatile liquid engaged therewith. The container has a reservoir portion in the form of a glass bottle containing a volatile liquid 3 and a wick 6 extending into the bottle. The wick 6 also extends above the top of the bottle through a seal and into a chimney means of the device 1. The wick 6 may be substantially cylindrical. The seal is present to retain the liquid 3 within the bottle should the device 1 be knocked over and/or inverted when the container is engaged therewith.

The device 1 has a housing 2 which partially extends over the container and its upper part. From the rear wall of the housing extends electrical plug formations 5.

The top of the housing 2 has a generally circular central aperture which defines the upper aperture of the chimney means. The upper aperture is aligned with a co-axial lower aperture (not shown), thus defining a channel therebetween for volatilised liquid to flow up and out of the upper aperture into the environment surrounding the device 1.

The emanation means may be provided in the form of at least one heater means and/or at least one electric fan. In FIGS. 1 & 2, the emanation means is illustrated in the form of a first electrical heating means 8 and a second electrical heating means 9. Said heating means 8,9 are illustrated as separate resistors, such as positive temperature coefficient (PTC) thermistors. However, either of both of the first or second electrical heating means 8,9 could be provided by way of a ring heater or the like, or a combination thereof.

An electrical fan 4 may also be provided. All of the fan 4 and the first and second electrical heating means 8,9 are in operable communication with a controller (not shown) which may act as the principal receiver of information from the motion sensor means 12. The controller processes the provided information and directly the control of the aforementioned components.

The device 1 may also be provided with at least one odour sensor (not shown) that is in operable communication with the controller.

Although not illustrated, the device 1 may be provided with means to receive at least two separate containers of liquid 3. In this arrangement the device 1 may be provided with additional emanation means to cause the emanation of the liquid 3, or a single set of emanation means, such as the first heater means 8 and second heater means 9 and the electric fan 4 could be operable to emanate liquid 3 from both containers as directed by the controller.

The mode of operation of the device and the inter-relation of the components will now be explained.

The device 1 must first be placed in an operational mode. There may be a user-activated switch (not shown) to permit the device to be switched into the operational mode. The device 10 will draw power from the power source which is depicted as plug formations 5 to draw mains electric power, this could be from solar cells mounted on the device and/or one or more batteries however.

In one mode, initiating the operational mode will cause the first heater means 8 to warm up to a temperature that will cause a standard amount of evaporation of the volatile liquid from the device 1, so called 0-50% duty cycle. If no motion or odour, or at least not greater than a base level of motion or odour, is detected from the motion sensor means 12 and/or the odour sensor means, the controller may direct the emanation means to continue to evaporate a standard amount of volatile liquid or begin a rest period, a so-called 0% duty cycle.

After a period of time set by the user or predetermined in the device, the sensor may be operative to inform the controller of the current movement and/or odour in the vicinity of the device such that the controller can direct a different response from the emanation means if there is a change.

The sensor(s) may in any event be operable to continually scan the vicinity of the device to inform the controller of a difference in the level of movement and/or odour. Alternatively the sensor(s) may only perform a scan at set intervals in order to conserve power consumption.

If the controller is informed of increased levels of motion and/or odour, the controller may direct the emanation means to emanate an increased quantity of liquid 3. For instance, when the input to the controller from the sensor(s) is indicative of a moderate amount of movement and/or odour in the nearby vicinity, the controller may instruct either or both heater means 8,9 to impart a standard amount of heat and also instruct the fan 4 to activate. Alternatively, the controller may instruct either or both heater means 8,9 to impart a greater than standard amount of heat (e.g. a 50-80% duty cycle).

Should the input from the sensor(s) to the controller be indicative of a large amount of movement and/or odour, the controller may instruct either or both heater means 8,9 to impart a greater amount of heat towards the wick than the standard amount (e.g. a 50-80% duty cycle) and instruct the fan 4 to activate. Alternatively, the controller may instruct either or both heater means 8,9 to impart a maximum amount of heat (e.g. 80-100% duty cycle) and/or instruct the fan 4 to be activate.

The controller may also be configured to adjust the activation time the emanation means are activated for to alter the quantity of liquid 3 that is evaporated in response to the input received from the sensor(s).

The device 1 may be operable to not emanate any liquid until the sensor(s) inform the controller that at least some movement and/or odour has been detected.

Where the device 1 is provided with two separate containers of liquid 3, the controller may be operable to analyse the input from the sensor(s) to determine whether there is an increased amount of movement and/or odour in the vicinity of the device 1. Based on the information received from the sensor(s), the controller may then be operable to alternate between the sources of liquids 3 based on the amount of movement and/or odour detected.

For instance, where the two separate containers of liquid are chemically different, one being a fragrance and one being a deodorant, and the input from the sensor(s) is indicative of an amount of odour being present in the vicinity of the device, the controller may be operable to cause just the deodorant liquid to be emanated. Whereas when the input from the sensor(s) is indicative of an amount of motion being present in the vicinity of the device, the controller may be operable to cause just the fragranced liquid to be emanated.

Alternatively where the input from the sensor(s) is indicative of a small to moderate amount of motion and/or odour being present in the vicinity of the device, the controller may be operable to cause just the fragranced liquid to be emanated. Whereas when the input from the sensor(s) is indicative of a large amount of motion and/or odour being present in the vicinity of the device, the controller may be operable to cause just the deodorant liquid to be emanated or cause both the deodorant and fragranced liquids to be emanated.

The illustrated device 1 is shown having an indicator 11 which is provided in the form of an LED. The LED(s) may be operable to provide a visual indication of the function currently being performed by the device. For instance, the LED could indicate when the device is in an operational mode by emitting a constant light which is converted to a flashing operation when motion has been detected.

The indicator 11 may also be provided with an audio component (not shown) wherein this component is capable of giving an audible alert when a particular function is being performed and/or motion has been detected or the like.

Alternatively or additionally, a screen (such as an LCD screen) could be presented on a prominent part of the device 1 to provide a message to a user indicating the current functioning of the device 1. For instance such messages could include “ON”, “SENSING”, “MOTION DETECTED”, “RESTING”, “NORMAL MODE”, “DETECTION MODE”, “OFF”.

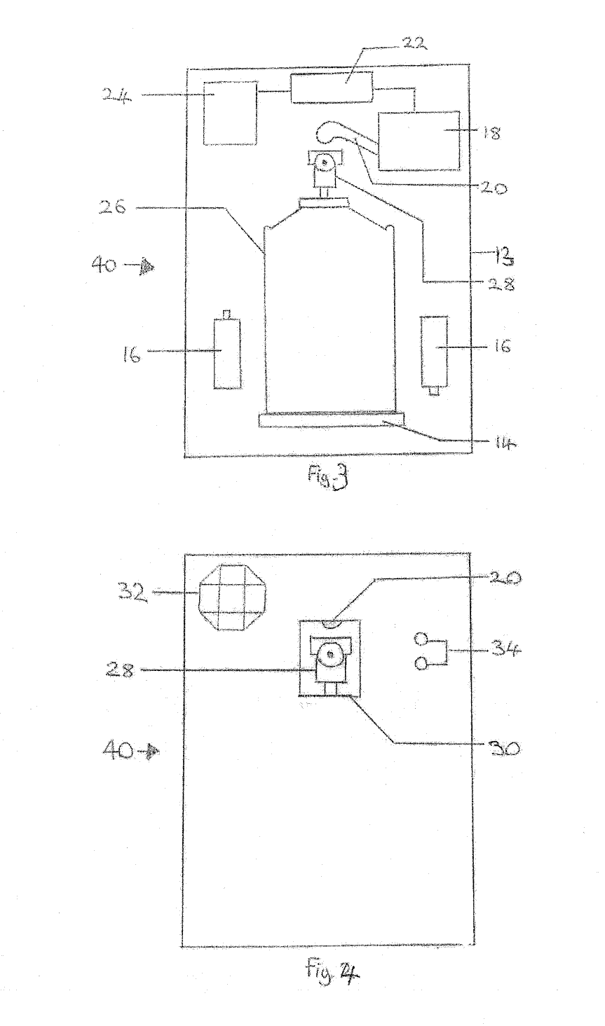

As shown in FIG. 3, the spraying device 40 comprises a housing 13 which supports a platform 14. The platform 14, in turn, is shaped to support and retain a container of fluid 26, such as an aerosol of fluid, when the device is in use. The housing 13 also supports an actuation means 18, a controller 22, a motion sensor means 24 and a power source which in FIG. 3 is depicted as a pair of batteries 16, although more or less batteries may be used. The housing may also support a odour sensor means (not shown) as well as or instead of the motion sensor means 24.

The device 40 is illustrated with a container of fluid 26 loaded therein and, specifically, FIG. 3 is depicted with an aerosol of fluid loaded therein. The fluid is sprayed from the can by an arm 20 connected to the emanation means 18 being moved in a downward direction and into contact with a spray head 28 of the aerosol. The movement of the arm 20 continues until the spray head is depressed and the valve within the aerosol is opened, thus, causing a quantity of fluid to be sprayed therefrom. Preferably the device 40 has a metered dose aerosol loaded therein. A metered dose aerosol being advantageous as a single depression of the spray head will release a predefined quantity of fluid from the aerosol regardless of the duration of time the spray head is depressed. However, a non-metered dose aerosol may be used in the device 40 as could a non-pressurised container possessing a pump mechanism to spray the fluid therefrom.

Alternatively, the emanation means 18 could take the form of a valve system, such as a solenoid valve system. Such a solenoid valve system may work together with a pressurised aerosol engaged therewith. Rather than initiate actuation by movement, the solenoid valve would be energised to initiate the release of a quantity of fluid from the aerosol.

Although not illustrated, the device 40 may be provided with means to receive at least two separate containers of fluid. In this arrangement the device 40 may be provided with additional emanation means 18 to cause the emanation of the fluid, or a single set of emanation means 18 to emanate fluid from both containers as directed by the controller.

Regardless of the specific form of the emanation means 18, the mode of operation of the device and the inter-relation of the components will now be explained in more detail.

The device 40 must first be placed in an operational mode. There will be a user-activated switch (not shown) to permit the device to be switched into the operational mode. The device 40 will draw power from the power source which may be mains electric power and/or solar cells mounted on the device, but is depicted in FIG. 3 as a pair of batteries 16.

The motion sensor means 24 and/or odour sensor means may also draw power from the batteries 16, either constantly or periodically, in order to sense for movement and/or odour in the vicinity of the device 40. If the sensor(s) is operable to communicate and input information to the controller 22. Once the controller 22 has received this information it is operable to instruct the emanation means 18 to emanate a quantity of fluid.

If no motion or odour, or at least not greater than a base level of motion or odour, is detected from the sensor(s), the controller 22 may direct the emanation means to continue to spray a standard quantity of fluid or may instruct the emanation means not to spray any fluid.

After a period of time set by the user or predetermined in the device, the sensor may be operative to inform the controller 22 of the current movement and/or odour in the vicinity of the device such that the controller 22 can direct a different response from the emanation means if there is a change.

The sensor(s) may in any event be operable to continually scan the vicinity of the device to inform the controller 22 of a difference in the level of movement and/or odour. Alternatively the sensor(s) may only perform a scan at set intervals in order to conserve power consumption.

If the controller 22 is informed of increased levels of motion and/or odour, the controller may direct the emanation means 18 to emanate an increased quantity of fluid. For instance, when the input to the controller from the sensor(s) is indicative of a moderate amount of movement and/or odour in the nearby vicinity, the controller may instruct the emanation means 18 to emanate a standard amount of fluid. A standard amount of fluid may be one dose of a metered dose aerosol or may be holding the valve of a standard aerosol open for one second.

Should the input from the sensor(s) to the controller be indicative of a large amount of movement and/or odour, the controller 22 may instruct the emanation means 22 to emanate a greater amount of fluid, such as two doses or holding the valve open for two seconds.

Where the device 40 is provided with two separate containers of fluid, the controller may be operable to analyse the input from the sensor(s) to determine whether there is an increased amount of movement and/or odour in the vicinity of the device. Based on the information received from the sensor(s), the controller may then be operable to alternate between the sources of fluids based on the amount of movement and/or odour detected.

For instance, where the two separate containers of liquid are chemically different, one being a fragrance and one being a deodorant, and the input from the sensor(s) is indicative of an amount of odour being present in the vicinity of the device, the controller 22 may be operable to cause just the deodorant fluid to be emanated. Whereas when the input from the sensor(s) is indicative of an amount of motion being present in the vicinity of the device, the controller 22 may be operable to cause just the fragranced fluid to be emanated.

Alternatively where the input from the sensor(s) is indicative of a small to moderate amount of motion and/or odour being present in the vicinity of the device 40, the controller 22 may be operable to cause just the fragranced fluid to be emanated. Whereas when the input from the sensor(s) is indicative of a large amount of motion and/or odour being present in the vicinity of the device, the controller may be operable to cause just the deodorant fluid to be emanated or cause both the deodorant and fragranced fluids to be emanated.

Turning to FIG. 42, the housing may be shaped such that it is capable of substantially completely surrounding a container of fluid 26. The front cover of the housing is illustrated and it can be seen that the front cover includes an aperture 30 therethrough which is in registration with the spray head 28 of the container 26. When the emanation means 18 cause the spraying of the fluid, the fluid exits the housing through the aperture 30 into the environment surrounding the device 40.

The motion sensor means 24 has a lens cover 32, which protrudes from the front of the device 40 to ensure a wide field of view. This may be advantageous where the motion sensor means is a passive infra-red sensor, since the motion would not need to be directly in front of the sensor means to be detected. Similarly, where the motion sensor means 24 is additionally or alternatively provided as a laser sensor or a flickering light sensor, this will also afford such sensors a wide field of view.

The lens 32 is located on, or protrudes through, an upper portion of the front cover of the housing in a position remote from the aperture 30. This ensures that the lens 32 is spaced away from and above the aperture which may prevent sprayed fluid erroneously coming into contact with the lens 32, thus preventing the lens 32 from becoming clouded with fluid over time.

The odour sensor means (not shown) may be positioned at any convenient location within the device. Preferably the odour sensor is provided in registration with an aperture through the housing 13 to more conveniently permit the detection of odours in the vicinity of the device 40.

Similarly, where the motion sensor means 24 incorporates, or comprises solely, a sound sensor, a port or aperture or microphone for receiving audio may also be positioned on an upper portion of the front cover of the housing in a position remote from the aperture 30 to prevent the sensor from coming into contact with sprayed fluid which may impair the sensing properties of said sensor.

The illustrated device 40 is shown having an indicator 34 which is provided in the form of a pair of LEDs. The LEDs may be operable to provide a visual indication of the function currently being performed by the device. For instance, the LEDs could indicate when the device is in an operational mode by emitting a constant light which is converted to a flashing operation when motion has been detected.

The indicator 11 may also be provided with an audio component (not shown) wherein this component is capable of giving an audible alert when a particular function is being performed and/or motion has been detected or the like.

Alternatively or additionally, a screen (such as an LCD screen) could be presented on a prominent part of the device 1 to provide a message to a user indicating the current functioning of the device 1. For instance such messages could include “ON”, “SENSING”, “MOTION DETECTED”, “RESTING”, “NORMAL MODE”, “DETECTION MODE”, “OFF”.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. An emanation device comprising:
a housing adapted to receive a container of fluid;
sensor means provided by at least one motion sensor means and/or at least one odour sensor means;
emanation means adapted to emanate, in use, the fluid;
a controller in communication with said sensor means and said emanation means;
wherein the controller is adapted to:
analyse the input from the sensor means;
make a determination of the quantity of fluid to emanated in response to said input;
instruct the emanation means to emanate the determined quantity of fluid.

2. An emanation device comprising:
a housing adapted to receive a container of fluid;
sensor means provided by at least one motion sensor means and at least one odour sensor means;
emanation means adapted to emanate, in use, the fluid;
a controller in communication with said sensor means and said emanation means;
wherein the controller is adapted to analyse the input from the sensor means and control the emanation means to emanate the determined amount of fluid.

3. An emanation device comprising:
a housing adapted to receive a container of fluid;
sensor means provided by at least one motion sensor means and/or at least one odour sensor means;
emanation means adapted to emanate, in use, the fluid;
a controller in communication with said sensor means and said emanation means;
wherein the controller is adapted to control the emanation means to emanate fluid only when the controller has received an input from the sensor means that is indicative of movement and/or odour in the vicinity of the device.

4. An emanation device comprising:
a housing adapted to receive at least two separate fluid sources;
sensor means provided by at least one motion sensor means and/or at least one odour sensor means;
at least one emanation means adapted to emanate, in use, the fluid;
a controller in communication with said sensor means and said emanation means;
wherein the controller is adapted to:
analyse the input from the sensor means;
make a determination of which source of fluid and/or the quantity of said fluid source to be emanated in response to said input;
instruct the emanation means to emanate the determined quantity of fluid.

5. An emanation device according to claim 1, wherein the controller is operative to provide an increased amount of emanation of fluid in response to the input from the sensor means informing the controller of an increased amount of movement and/or odour in the vicinity of the device.

6. An emanation device according to claim 5, wherein the input from the sensor means is analysed by the controller relative to one or more base levels.

7. An emanation device according to claim 6, wherein the base level is:
calculated by the device after being operated in a particular environment for a period of time; and/or
calculated such that the device is able modify the base level over time relative to the input it receives from the sensor means over time; and/or
a pre-determined value that is stored by the controller; and/or
controlled by a user.

8. An emanation device according to claim 1, wherein the motion sensor means is provided in the form of at least one of:
an infrared (IR) sensor; a laser sensor; and a sound sensor.

9. An emanation device according to claim 1, wherein the odour sensor means is operable to detect at least one of:
kitchen malodour; bathroom malodour; tobacco smoke; pet odours; mould and/or mildew; body odour; fish; onions; garbage; fragrance from other domestic products.

10. An emanation device according to claim 9, wherein the odour sensor means is adapted to detect at least some of the following chemical components: amines and nitrogen compounds; acids and/or sulphur compounds.

11. An emanation device according to claim 4, wherein the two or more fluid sources are different to each other.

12. An emanation device according to claim 4, wherein the controller is adapted to instruct the emanation means to alternate between the sources of fluids based on the amount of movement and/or odour detected.

13. An emanation device according to claim 4, wherein the device is provided with at least one motion sensor means and at least one odour sensor means, and the controller is adapted to cause a first source of fluid to be emanated in response to an input received from the odour sensor(s) and optionally is adapted to cause a second source of fluid to be emanated in response to an input received from the motion sensor(s).

14. An emanation device according to claim 2, wherein the controller is operative to provide an increased amount of emanation of fluid in response to the input from the sensor means informing the controller of an increased amount of movement and/or odour in the vicinity of the device.

15. An emanation device according to claim 14, wherein the input from the sensor means is analysed by the controller relative to one or more base levels.

16. An emanation device according to claim 3, wherein the controller is operative to provide an increased amount of emanation of fluid in response to the input from the sensor means informing the controller of an increased amount of movement and/or odour in the vicinity of the device.

17. An emanation device according to claim 16, wherein the input from the sensor means is analysed by the controller relative to one or more base levels.

18. An emanation device according to claim 4, wherein the controller is operative to provide an increased amount of emanation of fluid in response to the input from the sensor means informing the controller of an increased amount of movement and/or odour in the vicinity of the device.

19. An emanation device according to claim 18, wherein the input from the sensor means is analysed by the controller relative to one or more base levels.
